(19) United States
(12) Patent Application Publication
(10) Pub. No.: US 2010/0232673 A1
(43) Pub. Date: Sep. 16, 2010

(54) REDUCTION OF ARTIFACTS CAUSED BY MOVEMENT OF AN X-RAY TUBE IN OBJECT RECONSTRUCTION

(76) Inventors: Benjamin Keck, Ammerndorf (DE); Markus Kowarschik, Langenzenn (DE); Jasmina Ludwig, Munchen (DE); Thomas Mertelmeier, Erlangen (DE); Holger Scherl, Erlangen (DE)

Correspondence Address: BRINKS HOFER GILSON & LIONE, P.O. BOX 10395, CHICAGO, IL, 60610, US

(21) Appl. No.: 12/706,894

(22) Filed: Feb. 17, 2010

(30) Foreign Application Priority Data
Feb. 19, 2009 (DE) 10 2009 009 617.5

(51) Int. Cl. G06K 9/00; A61B 6/03

(52) U.S. Cl. 382/132; 378/62

(57) ABSTRACT

An improvement in image quality when determining an image using iterative reconstruction is provided by taking account of focus shifts. In order to take into consideration the influence of focus shift in calculated projections, several different projections are calculated for each section of the focus path on a section-by-section basis. The several different calculated projections for a section are averaged for comparison with a projection recorded in the section. Improved image quality is provided, for example, in tomosynthesis applications in which a large number of recordings are made with a stationary detector.

This application claims the benefit of DE 10 2009 009 617.5, filed Feb. 19, 2009, which is hereby incorporated by reference.

### BACKGROUND

The present embodiments relate to a method and an apparatus for improving image quality when determining an image using iterative reconstruction.

In medical diagnostics, an object to be examined is irradiated with x-ray radiation. Attenuation of the x-ray radiation takes place during penetration of the object. After penetrating the object, the attenuated radiation is captured by a detector.

The attenuation of the x-ray radiation through the object represents a measure of the density of an irradiated material (e.g., the object). In medical diagnostics, the attenuation and/or density represented in the form of images suggests the presence of abnormalities and/or tumors.

In one individual recorded two-dimensional x-ray image, information about a three-dimensional object is obtained. The obtained information about the three-dimensional object is sometimes insufficient for tumor tissue to be identified. The overlapping of healthy tissue can lead to diagnostic errors.

In individual recorded x-ray images, the recording provides information about the overall attenuation of x-rays along a path of the x-rays through the object. A pixel value of the detector therefore corresponds with an integration over the attenuation value or attenuation coefficient along a path extending through the object.

For a more reliable diagnosis, it is desirable not only to obtain integrations over the attenuation value, but also to obtain the attenuation value itself as a function of the position (e.g., the attenuation coefficient as a scalar, position-dependent field). In order to identify the attenuation value in three dimensions, several recordings can be made, from which the attenuation coefficient is determined. Such a set of attenuation coefficients obtained by reconstruction can also be viewed as a three-dimensional image data set and is also referred to below as an image data set or an image. The three-dimensional image data set generally includes several layer images.

The determination or reconstruction of the attenuation coefficient from the several recordings (i.e., projections) is a complex mathematical problem.

Various methods (e.g., listed in Buzug, Thorsten M., “Computed Tomography: From Photon Statistics to Modern Cone-Beam CT.” Springer, 2008) have been proposed in order to solve the complex mathematical problem. One group of methods is the iterative methods, or iterative reconstruction methods. In the iterative reconstruction methods, position-dependent attenuation values of the object, or the image, are specified as an initial approximation, and the projections resulting from the initial approximation are calculated. The calculated projections are compared with the measured projections (i.e., differentiation). On the basis of the comparison, a correction of the image is performed. A new calculation of projections takes place, which are compared with the measurement results. When there is a match between the calculated and measured projections within a requisite level of accuracy, the corresponding image is converged out and provides the desired attenuation coefficients.

In the iterative reconstruction method, projections are obtained from one image, and differences between projections (e.g., calculated and measured) are used for the correction of the image. For this purpose, operations are performed in the image space (e.g., position-dependent attenuation coefficients of the object) and in the projection space (e.g., representations of the x-ray radiation attenuated by the object obtained for various projection directions). The calculation of projections for an image is referred to as forward projection, and the calculation of an image or an image correction from projections or differences between projections is referred to as back projection.

In the iterative reconstruction method, errors can occur in the reconstructed image. The errors are caused, for example, by incomplete data, reorganization of images (e.g., rebinning) or focus shift.

The generation and focusing of x-ray radiation is typically performed in an x-ray tube. The x-ray tube generally generates a cone-shaped beam (i.e., cone beam) or a fan-shaped beam (i.e., fan beam). The exit point of the beam is the focus or focal point in the x-ray tube at which the x-rays are concentrated.

The reconstructed image can include qualitative defects for several reasons. The specialist literature also refers to the qualitative defects as artifacts. Reasons for the artifacts can include, for example, that the information is incomplete in numerical terms or also in terms of focus shift. The medical imaging technique known as tomosynthesis can be particularly strongly affected by the problem of artifacts. Tomosynthesis is used, for example in digital mammography. In contrast to computed tomography, tomosynthesis is based on the principle that a comparatively small angular interval is scanned as the x-ray tube moves about the object to be examined. The limit on the interval is generally determined by the object to be examined (e.g., a female breast).

A sequence of tomosynthesis projections in mammography may be recorded using a modified mammography system or a breast tomosynthesis system. Thus, for example, 25 projections may be generated as the x-ray tube moves above the detector in an angular range between −25° and 25°. During the movement of the x-ray tube, the radiation is triggered at regular intervals and is read out of the detector for each projection. In a subsequent tomosynthesis reconstruction process, which proceeds as described above, a three-dimensional representation of the examined object or of the image is reconstructed in the computer. During the medical assessment, the reconstructed layer images that are oriented parallel to the detector level (e.g., Z layers) are generally taken into consideration.

In order to achieve mechanical stability in the system, the movement of the x-ray tube is performed continuously (e.g., no step-and-shoot process). The x-ray tube remains in continuous motion while, at the same time, the radiation is triggered (see, Bissonnette, M, et al., “Digital breast tomosynthesis using an amorphous selenium flat panel detector.” Proc. SPIE, 5745, 529-40 (2005)). The consequence of the continuous motion is that the imaging of the internal structures of the imaged object is blurred in the projection images (see, Ren, Baorui, et al., “Design and performance of the prototype full field breast tomosynthesis system with selenium based flat panel detector.” Proc. SPIE Physics of Medical Imaging, 5745, 550-61 (2005); Zhao, Bo and Wei Zhao, “Imaging performance of an amorphous selenium digital mammography detector in a breast tomosynthesis system.” Med. Phys., 35.5, 1978-87 (2008)). Following the conventional reconstruction process, the blurring of the internal structures of the imaged object is reflected in the layer images in the form of a fuzzy presentation (e.g., blurring artifacts).

The blurring artifacts, which are caused by the movement of the x-ray tube, are particularly pronounced in imaging systems in which no synchronous isocentric movement of the x-ray tube and an image receiver or the detector takes place. This is the case, for example in a tomosynthesis system with a stationary detector. The degree of blurring is also a function of the pulse duration of the x-ray tube, which is in turn a function of the initial dose and thus the thickness of the breast. The greater the pulse duration, the longer the route which is taken by the x-ray tube per radiation pulse, and thus the greater the blurring of the object structures.

In the case of very small anatomical structures (e.g., in mammography, the microcalcifications that are relevant for the assessment) the blurring of the object structures can be highly critical. The blurring can lead to the individual microcalcifications in the image becoming merged, or the contrast of the microcalcifications in the reconstructed layers being reduced. This can lead to incorrect diagnoses.

### SUMMARY AND DESCRIPTION

The present embodiments may obviate one or more of the drawbacks or limitations in the related art. For example, in one embodiment, image quality in the case of image determination through the use of iterative reconstruction may be improved. The present embodiments concentrate on artifacts in iterative methods caused by focus shift.

In the following, iterative reconstruction may be defined as an image reconstruction from measured projections, in which a comparison is performed between calculated and measured projections in order to align the image with the measured projections. The term “iterative” may be defined in that the method may repeat the comparison between the calculated and measured projections if the image does not satisfy quality requirements, or a convergence criterion is not fulfilled. However, the term “iterative” is not intended to exclude an arrangement in which the first comparison yields an image that satisfies the requirements (e.g., borderline cases of an iteration are not excluded).

An iterative reconstruction method may include repeated execution of the following acts: Estimating the starting volume (e.g., initial approximation of the three-dimensional representation of the object or image); calculating the forward projection for each angular position of the x-ray tube; comparing the calculated projections with the measured projections by use of differentiation and determining therefrom a correction projection (e.g., one correction projection per angular position); correcting the actual volume by back projection of the differentiated images; and repeating the calculating, the comparing, and the correcting.

In one embodiment, all forward projections may not be calculated before the differentiation and subsequent back projection of the differentiated images. A projection-by-projection approach may instead be taken. One embodiment may include additional acts, such as, for example, filtering acts (e.g., low-pass filtering of the image after back projection of all differentiated projections).

The present embodiments improve image quality by taking account of the movement of an x-ray source or a focus of the x-ray source in the calculation (e.g., forward projection). The present embodiments may be employed in situations where iterative reconstruction is used (e.g., in applications in the area of tomosynthesis or computed tomography (CT)).

In one embodiment, a section of a path passed through by the focus of the x-ray source during recording of a projection is determined. Several different projections are calculated for the section of the path. An averaging of the several different projections is performed for a comparison of the averaged projection with the recorded projection.

In one embodiment, the entire focus path is subdivided into sections or segments, for each of which a plurality of projections are calculated and compared with a measured projection.

In addition to a method, an apparatus realized or configured to perform the method also forms the subject matter of the present embodiments. The embodiments of the apparatus may be realized using software, hardware (e.g., a processor, a memory), firmware or a combination thereof. In one embodiment, the apparatus may include functional modules that realize one or several process acts.

### BRIEF DESCRIPTION OF THE DRAWINGS

FIG. 1 shows a lateral view of one embodiment of a mammography device,

FIG. 2 shows a frontal view of one embodiment of the mammography device of FIG. 1,

FIG. 3 shows two deflection positions during irradiation through the use of a mammography device during tomosynthesis,

FIG. 4 shows a method for the iterative reconstruction of an image,

FIG. 5 shows one embodiment of a process for improving image quality when determining an image using iterative reconstruction,

FIG. 6 shows the method of FIG. 4 modified for a process according to one embodiment of the process shown in FIG. 5,

FIG. 7 shows an initialization for one embodiment of the method of FIG. 6.

### DETAILED DESCRIPTION

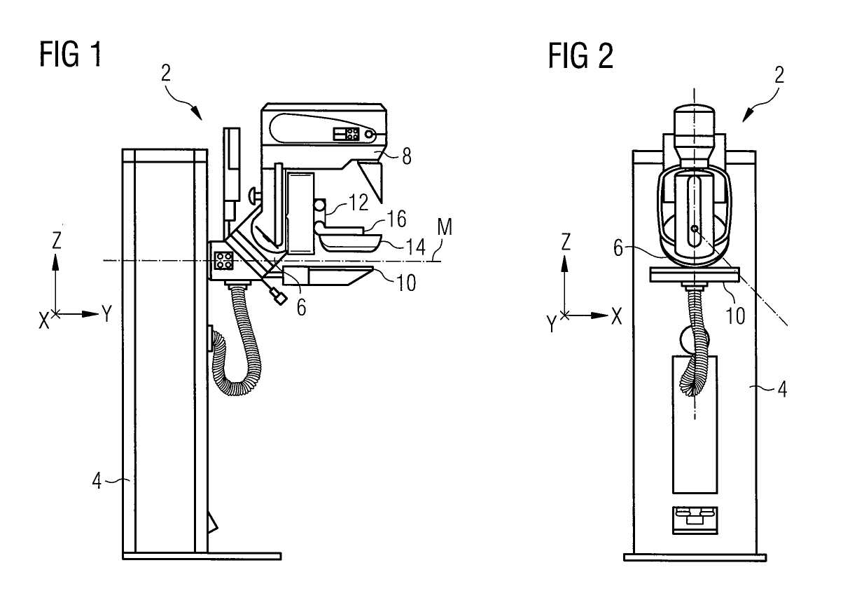

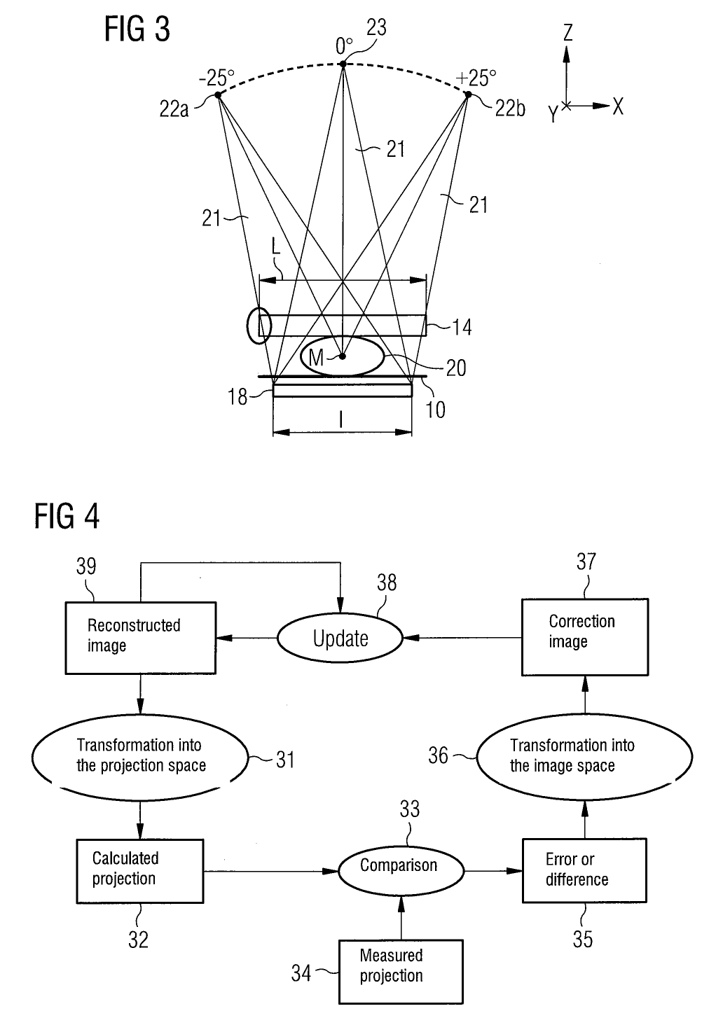

FIG. 1 and FIG. 2 show a lateral view and a frontal view, respectively, of one embodiment of a mammography device 2. The mammography device 2 has a main body (e.g., a stand 4) and a device arm 6 that projects and is angled from the stand 4. At a free end of the device arm 6, a radiation unit 8 (e.g., an x-ray emitter) is arranged. An object couch 10 and a compression unit 12 are also mounted on the device arm 6. The compression unit 12 may include a compression element 14 arranged movably along a vertical Z direction relative to the object couch 10, and a mount 16 for the compression element 14. The compression unit 12 includes a lifting assembly to move the mount 16, and thus the compression element 14. A detector 18 (e.g., a digital detector) is arranged in a lower area of the object couch 10, as shown in FIG. 3.

The mammography device 2 may be provided for tomosynthesis examinations (e.g., a tomosynthesis system), in which the radiation unit 8 is moved over an angular range about a central axis M that runs in parallel to the Y direction, as can be seen in FIG. 3. Thus, several projections of an object 20 to be examined are obtained, the object being held in a fixed position between the object couch 10 and the compression element 14. During the recording of images from the different angularities, an x-ray 21 with a conical or fan-shaped cross section penetrates the compression element 14, the object 20 to be examined, and the object couch 10 and strikes the detector 18. In one embodiment, the detector 18 may be sized such that the captured images may be made in the angular range between two deflection positions 22a, 22b with corresponding deflection angles of −25° and/or +25°. The deflection positions 22a, 22b are arranged in the X-Z plane on both sides from a zero position 23, in which the x-ray 21 strikes the detector 18 vertically. In one embodiment, the planar detector 18 may measure 24×30 cm.

In one embodiment, twenty five recordings may be made while passing through the path from the deflection position 22a to the deflection position 22b. From the recorded projections, one image is determined iteratively for the examined object 20.

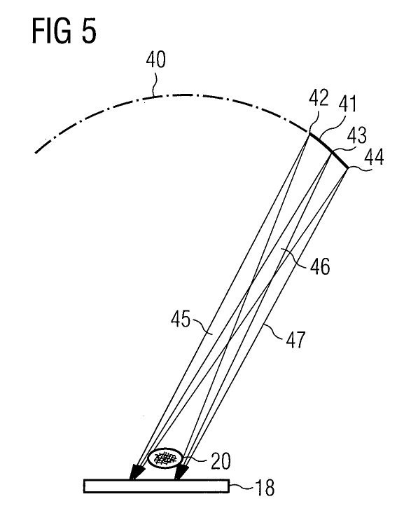

FIG. 4 schematically shows an iterative method for a reconstruction of an image from the measured projections. The image or corresponding attenuation values for the object is/are initialized with initial values. On the basis of the initial approximation, a projection 32 is calculated in the course of a forward projection 31. In act 33, the calculated projection 32 is compared with a measured projection 34. The difference or error 35 is determined. The error determination is made for all projections. The difference between the calculated projection 32 and the measured projection 34 is transformed into an image space in the course of a back projection 36. A correction image 37 is obtained, which is linked in an update 38 with the current image to form a new image 39. In one embodiment, the result of the transformation into the image space 36 is allocated directly to the current approximation for the reconstruction result to give a new approximation or a new image. The new image 39 or updated image is then used once again for the calculation of projections by use of forward projection 31.

The method is completed when there is a sufficient match between the calculated projection 32 and measured projection 34. One embodiment may use a convergence criterion to determine when there is a sufficient match between the calculated projection 32 and the measured projection 34.

During recording of the projections, an x-ray source passes through a path in order to capture recordings from different angles. The movement of the x-ray source is generally not interrupted for the recordings, and instead, there is continuous movement at a constant velocity from the start of the path until the end. The individual projections therefore originate not from a stationary source but instead from a moving source. On account of the finite pulse duration or recording duration of a projection, a focus of the x-ray source passes through a path section during recording (e.g., a focus shift).

In the existing methods, one projection is calculated for each angular position. By approximation, a stationary x-ray source with a point-shaped (e.g., infinitely small) focus is assumed. The continuous movement of the x-ray source is not taken into consideration. As a consequence of mechanical instabilities of the tomosynthesis system, which are not ideal, a scanning path of the x-ray source may be modeled using projection matrices that depict the perspective imaging of a three-dimensional object space (e.g., the image space) into a two-dimensional projection space. The projection matrices may be determined in advance during calibration of the tomosynthesis system.

In one embodiment, the focus shift is taken into consideration in that not just one, but several projections are calculated for the section of the focus path, or the x-ray source that corresponds to the measured projection. The calculated projections are averaged, and the averaged projections are used for the comparison with the measured projection.

The entire focus path may be divided into a plurality of sections or segments, which are each assigned to a measured projection. Several projections are calculated and averaged for each of the plurality of segments.

One embodiment of this process described above is illustrated in FIG. 5. A focus path 40 for a tomosynthesis recording is shown with a mostly dashed line. In one embodiment, the tomosynthesis recording is a recording with a tomosynthesis system in digital mammography with a stationary detector (e.g. a recording with the embodiments shown in FIGS. 1-3). As shown in FIG. 4, the x-ray source is moved on an arced path (e.g., 50°, radius 590 mm). In one embodiment, the entire scan takes 12.7 sec with a pulse duration of 50 ms; the length of a segment of the circuit (e.g., a focal path length) is then approx. 2 mm.

As shown in FIG. 5, one segment 41 of the focus path 40 is indicated by a continuous line. The detector 18 records a projection of the object 20 (e.g., a female breast in the case of a mammography examination) while the focus passes through the segment 41. The detector 18 is stationary during the recording. A radiation cone 45, 46, 47 is indicated for each of three focus positions 42, 43, 44 during recording of the projection. Projections are calculated for the three focus positions 42, 43, 44 or x-rays 45, 46, 47. Instead of the projection calculation that usually occurs for a focus position 46, which is generally central relative to the segment, a calculation of the corresponding projection is performed for the three focus positions 42, 43, 44. The three results are averaged. Each calculated projection may be weighted (e.g., by a factor of ⅓). A different weighting (e.g., greater weighting of the central focus position 46) may also be used. An optimal number of calculated projections per segment and the weightings assigned may be determined, for example, with the aid of tests on a phantom.

In one embodiment, the number of calculated projections and projections to be averaged per segment may vary as a function of the position of the segment on the scanning path. In the case of tomosynthesis, the segments positioned slightly further out may be scanned more precisely, since the segments positioned slightly further out contribute to a greater blurring of the object structures. In one embodiment, a non-uniform dose distribution (e.g., different pulse lengths) during the scan, resulting from segments of differing lengths, may be taken into consideration. The number of focus positions assigned per segment for the projections to be calculated and averaged may be variable by segment.

In one embodiment, the segment-specific numbers of focus positions may be selected as a function of the iteration. In order to achieve, at the start of the iterative calculation, a usable approximation for the three-dimensional representation of the object in a computationally-efficient manner, a relatively rough scan of the segments may be made. The usable approximation may then be improved using finer scans of the segments in the course of subsequent iterations.

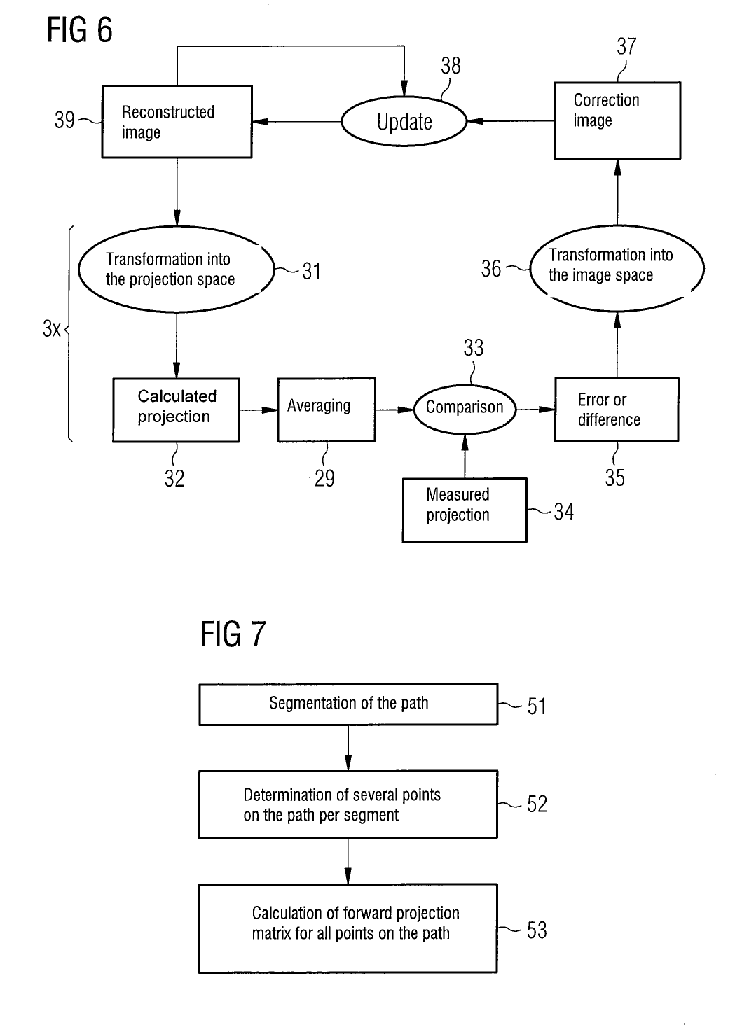

FIG. 6 shows how the present embodiments affect the process shown in FIG. 4. Projection calculation 31 is performed several times for each segment for different points on the path. Accordingly, several calculated projections 32 are obtained (e.g., three for the segment 41 shown in FIG. 5). The several calculated projections 32 are averaged in act 29 for comparison 33 with a corresponding measured projection 34.

As shown in FIG. 7, initial values may be calculated before the scan to allow for rapid image determination. The projection matrices required for the forward projection may be calculated in advance (e.g., act 53) on the basis of the segmentation of the path (e.g., act 51) and the selection, by segment, of points on the path for the projection calculation (e.g., act 52). By calculating the initial values before the scan, the additional effort caused by multiple calculations of projections according to the present embodiment does not have as great an effect on the time taken for the calculation of the image. The matrices may be used for several scans provided that no parameters are changed in relation to the path and x-ray source.

The present embodiments are not restricted to the cases described above. The present embodiments may also, for example, be employed if the detector is not stationary, but instead moves (e.g., in the case of CT examinations).

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for the improvement of image quality when determining an image by iterative reconstruction comprising:
determining a section of a path passed through by a focus of an x-ray source during recording of a projection;
calculating several different projections for the section; and
averaging the several projections for a comparison of an averaged projection with a recorded projection.

2. The method as claimed in claim 1, wherein the averaging is a weighted averaging.

3. The method as claimed in claim 1 comprising subdividing the path into a plurality of sections, including the section, and calculating the several projections and averaging the several projections for each of the plurality of sections.

4. The method as claimed in claim 3, wherein the number of calculated projections for the section is determined according to the position of the section on the path.

5. The method as claimed in claim 3, wherein the lengths of the plurality of sections are determined according to a dose distribution.

6. The method as claimed in claim 1 comprising performing several iterations in which the comparison of the averaged projection with the recorded projection is performed, wherein the number of calculated projections for a section is determined according to an iteration number.

7. An apparatus for the improvement of image quality when determining an image by using iterative reconstruction comprising:
an x-ray source; and
a processor configured to determine a section of a path passed through by a focus of an x-ray source during recording of a projection, calculate several different projections for the section, and perform an averaging of the several projections for a comparison of the averaged projection with a recorded projection.

8. The apparatus as claimed in claim 7, wherein the processor is configured to perform a weighted averaging of the several projections.

9. The apparatus as claimed in claim 7, wherein the processor is configured to subdivide the path into a plurality of sections, and calculate the several different projections and perform an averaging of the several projections for each of the plurality of sections.

10. The apparatus as claimed in claim 9, wherein the processor is configured to determine the number of calculated projections for a section according to the position of the section on the path.

11. The apparatus as claimed in claim 9, wherein the lengths of the plurality of sections are determined according to a dose distribution.

12. The apparatus as claimed in claim 9, wherein the processor is configured to perform several iterations in which the comparison of the averaged projection with the recorded projection is performed, and determine the number of calculated projections for a section according to an iteration number.

13. The method as claimed in claim 2 comprising subdividing the path into a plurality of sections, including the section, and calculating the several projections and averaging the several projections for each of the plurality of sections.

14. The method as claimed in claim 4, wherein the lengths of the plurality of sections are determined according to a dose distribution.

15. The method as claimed in claim 2 comprising performing several iterations in which the comparison of the averaged projection with the recorded projection is performed, wherein the number of calculated projections for a section is determined according to an iteration number.

16. The method as claimed in claim 3 comprising performing several iterations in which the comparison of the averaged projection with the recorded projection is performed, wherein the number of calculated projections for a section is determined according to an iteration number.

17. The apparatus as claimed in claim 8, wherein the processor is configured to subdivide the path into a plurality of sections, and calculate the several different projections and perform an averaging of the several projections for each of the plurality of sections.

18. The apparatus as claimed in claim 10, wherein the lengths of the plurality of sections are determined according to a dose distribution.

19. The apparatus as claimed in claim 10, wherein the processor is configured to perform several iterations in which the comparison of the averaged projection with the recorded projection is performed, and determine the number of calculated projections for a section according to an iteration number.

20. The apparatus as claimed in claim 11, wherein the processor is configured to perform several iterations in which the comparison of the averaged projection with the recorded projection is performed, and determine the number of calculated projections for a section according to an iteration number.
